(19) United States
(12) Patent Application Publication
(10) Pub. No.: US 2009/0163919 A1
(43) Pub. Date: Jun. 25, 2009

(54) DEVICES, SYSTEMS, AND METHODS FOR DELIVERY OF A PHARMACEUTICAL TO A SUBJECT'S SPINE

(76) Inventors: Peter Tarcha, Lake Villa, IL (US); Abhijeet Joshi, Austin, TX (US); Brian Bergeron, Austin, TX (US); Diana Martinez, Austin, TX (US); Marc Peterman, Austin, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/960,445

(22) Filed: Dec. 19, 2007

(51) Int. Cl. A61B 17/86; A61F 2/44; A61B 17/56; A61L 27/54; A61L 27/58; A61B 17/80; A61B 17/58

(52) U.S. Cl. 606/70; 623/17.11; 606/301; 424/423; 424/426

(57) ABSTRACT

The present disclosure relates, according to some embodiments, to devices, systems, and/or methods for delivery (e.g., controlled delivery) of a pharmaceutical to a subject's spine. For example, the disclosure relates to implants that provide both a mechanical function and a pharmacological function. According to some embodiments, a spinal implant may comprise a rigid spinal implant body having one or more recesses, one or more layers in each of the one or more recesses, each of the one or more layers comprising a biocompatible material and a pharmaceutically effective amount of a pharmaceutical compound; and at least one biocompatible cap in each of the one or more recesses. An implant may further comprise biocompatible barriers interspersed between the layers.

### FIELD OF THE DISCLOSURE

The present disclosure relates, in some embodiments, to compositions, devices, systems, and methods for delivery of a pharmaceutical compound to a spine.

### BACKGROUND OF THE DISCLOSURE

The spine comprises vertebrae, intervertebral discs separating the vertebrae, the sacrum, and coccyx. Intervertebral discs comprise a tough, fibrous outer ring, called the annulus fibrosis, and a viscous, fluid-filled central core called the nucleus pulposus. In addition, the spine comprises a spinal canal that houses the spinal cord. The spinal canal is protected by the intervertebral foramen in the vertebral regions and by the ligamentum flavum and the posterior longitudinal ligament in the intervertebral spaces. The spinal cord is enclosed within meninges, which consists of three layers of connective tissue. Blood vessels and capillaries that supply blood to the spinal cord run through the meninges and the space inside the outer layer (membrane) of the meninges is filled with cerebrospinal fluid.

Spinal diseases and injuries continue to be among the most painful and dehabilitating, despite advances in the understanding of spinal physiology, neurophysiology, pathology, and trauma. Attempts to treat a spinal condition may be less effective than desired, in part, because of the unique attributes of the spinal environment. For example, the flexibility and range of motion of the spine may generate large intradiscal pressures during normal loading of the spine. These pressures may interfere with normal healing processes. Sutures in connective tissues of the spine (e.g., annulus fibrosis, meninges) may pull out quickly, may aggravate existing tears and/or may nucleate new tears. In addition, drug delivery may be difficult since areas of the spine (e.g., annulus fibrosis) lack a direct blood supply.

### SUMMARY

Accordingly, a need has arisen for improved compositions, devices, systems, and methods for delivery of a pharmaceutical compound to a spine.

The present disclosure relates, according to some embodiments, to compositions, devices, systems, and/or methods for delivery (e.g., controlled delivery) of a pharmaceutical to a subject's spine.

A spinal implant may comprise, according to some embodiments, a rigid spinal implant body having one or more recesses, one or more layers in each of the one or more recesses, each of the one or more layers comprising a biocompatible material and a pharmaceutically effective amount of a pharmaceutical compound, and at least one biocompatible cap in each of the one or more recesses. Each recess may independently have a shape selected from, for example, cube, hemisphere, cone, polyhedron, pyramid, and/or cylinder. A recess may be defined, for example, by a base and lateral walls and may have an aperture. In some embodiments, a recess may include two or more layers (e.g., with a barrier in between). A recess may have, for example, an aperture, one or more lateral walls, and a base with a structure as follows:

biocompatible cap—(layer—biocompatible barrier)ₙ—layer—RECESS BASE,

wherein n may be an integer from about 1 to about 50 (e.g., 2 to about 50) and RECESS BASE is the base of the recess. The composition of each layer (e.g., choice of biocompatible material and/or choice of pharmaceutical agent) may be the same or different, independently.

According to some embodiments, each biocompatible barrier may be biodegradable at a rate successively faster than the next lower biocompatible barrier closer to the base of the recess (e.g., an outermost barrier biodegrades more rapidly than a middle barrier and the middle barrier biodegrades more quickly than an innermost barrier. A barrier, independent of its biodegradation rate relative to other barriers, may biodegrade at a faster rate than one or more layers in some embodiments.

A rigid spinal implant body, according to some embodiments, may comprise a pedicle screw, a polyaxial screw, an interbody spacer, an anterior cervical plate, an open hook, a rod, a rod-to-rod connector, a cable, a cam, a bone plate, a bone screw, a vertical endplate, a cage, an artificial disc, and combinations thereof.

In some embodiments, a recess may be from about one millimeter to about twenty millimeters in its longest transverse dimension (e.g., diameter). A recess may be from about 0.5 millimeters to about 20 millimeters deep according to some embodiments. Depth and transverse dimension may be independent or interdependent (e.g., proportional).

A biocompatible material, in some embodiments, may comprise a polymer. A polymer may comprise, for example, macromolecule comprising a pendant phosphorylcholine group. A polymer may comprise, for example, a material having the formula poly(MPC_{w}:LMAₓ:HPMA_{y}:TSMA_{z}) wherein MPC is 2 methacryoyloxyethylphosphorylcholine, LMA is lauryl methacrylate, HPMA is hydroxypropyl methacrylate, TSMA is trimethoxysilylpropyl methacrylate, and w, x, y, and z are independently integers from 0 to 60. A polymer may comprise, for example, a compound selected from a phosphorylcholine linked macromolecule, an oligoethylenimine, a polyethylenimine, and combinations thereof.

According to some embodiments, a pharmaceutical compound may comprise a compound selected from an adhesive, an arterial vessel wall irritant, a bone morphogenic protein, an extracellular matrix component, an inflammatory cytokine, a polymer, and combinations thereof. A pharmaceutical compound may comprise a compound selected from an analgesic, an antimicrobial agent, an anti-inflammatory agent, a fibrosis-inducing agent, and combinations thereof in some embodiments.

The present disclosure relates, in some embodiments, to a method of delivering a pharmaceutically effective amount of a pharmaceutical compound to a subject's spine, said method comprising (a) implanting in the subject's spine a spinal implant comprising: (1) a rigid spinal implant body having one or more recesses, (2) one or more layers in each of the one or more recesses, each of the one or more layers comprising a biocompatible material and a pharmaceutical compound, wherein the layer is configured and arranged to deliver a pharmaceutically effective amount of the pharmaceutical compound to the subject's spine, and (3) at least one biocompatible cap in each of the one or more recesses. According to some embodiments, a spinal implant for delivering a pharmaceutical may further comprise two or more layers in each of the one or more recesses. At least one biocompatible barrier may be positioned between the at least two layers. Each of the one or more recesses of an implant for delivering a pharmaceutical, in some embodiments, may comprise an aperture, one or more lateral walls, and a base and wherein the two or more layers, the at least one biocompatible barrier, and the at least one biocompatible cap biocompatible have the structure: biocompatible cap—layer—biocompatible barrier)n—layer—RECESS BASE, wherein n may be an integer from about 1 to about 100 (e.g. from 2 to about 50) and RECESS BASE is the base of the recess. According to some embodiments, each biocompatible layer may have the same composition. Some biocompatible layers may have a first composition and other layers in the same recess or different recesses may have a second composition in some embodiments.

According to some embodiments for delivering a pharmaceutical, each biocompatible barrier may be biodegradable at a rate successively faster than the next lower biocompatible barrier closer to the base of the recess (e.g., an outermost barrier biodegrades more rapidly than a middle barrier and the middle barrier biodegrades more quickly than an innermost barrier. A barrier, independent of its biodegradation rate relative to other barriers, may biodegrade at a faster rate than one or more layers in some embodiments.

A rigid spinal implant body for delivering a pharmaceutical, according to some embodiments, may comprise a pedicle screw, a polyaxial screw, an interbody spacer, an anterior cervical plate, an open hook, a rod, a rod-to-rod connector, a cable, a cam, a bone plate, a bone screw, a vertical endplate, a cage, an artificial disc, and combinations thereof.

In some embodiments, a recess of an implant for delivering a pharmaceutical may have the same or different dimensions as a recess of an implant used for other purposes (e.g., delivering other materials). Similarly, an implant for delivering a pharmaceutical may have biocompatible materials that are the same or different dimensions as a recess of an implant used for other purposes (e.g., delivering other materials).

According to some embodiments, a pharmaceutical compound may comprise a compound selected from an adhesive, an arterial vessel wall irritant, a bone morphogenic protein, an extracellular matrix component, an inflammatory cytokine, a polymer, and combinations thereof. A pharmaceutical compound may comprise a compound selected from an analgesic, an antimicrobial agent, an anti-inflammatory agent, a fibrosis-inducing agent, and combinations thereof in some embodiments.

According to some embodiments, the present disclosure relates to a method of manufacturing a spinal implant comprising (a) providing a rigid spinal implant body having one or more recesses, (b) depositing in each of the one or more recesses at least one layer comprising a biocompatible material and a pharmaceutical compound, wherein the layer is configured and arranged to deliver a pharmaceutically effective amount of the pharmaceutical compound to the subject's spine, and (c) depositing in each of the one or more recesses at least one biocompatible cap over the one or more layers obturating the recess, wherein each biocompatible cap is configured and arranged to biodegrade at a slower rate than the layer it covers. Depositing may include, for example, printing the one or more layers. The configuration and arrangement (e.g., size, shape, layer composition, barrier composition, degradation rate) of each recess manufactured may be independently the same or different from as otherwise described herein.

In some embodiments, the present disclosure relates to an implantable bone screw (e.g., a pedicle screw, a polyaxial screw) comprising a rigid implantable bone screw body having bone screw threads and a bone screw head having one or more recesses, two or more layers in each of the one or more recesses, each of the two or more layers comprising a biocompatible material and a pharmaceutically effective amount of a pharmaceutical compound, at least one biocompatible barrier positioned between the at least two layers, and at least one biocompatible cap in each of the one or more recesses. Each of the one or more recesses on an implantable bone screw comprises an aperture, one or more lateral walls, and a base and wherein the two or more layers, the at least one biocompatible barrier, and the at least one biocompatible cap have the structure: biocompatible cap—layer—biocompatible barrier)n—layer—RECESS BASE, wherein n may be an integer from about 1 to about 100 and RECESS BASE is the base of the recess.

The present disclosure relates, according to some embodiments, to an implantable bone plate (e.g., a cervical plate) comprising a rigid implantable bone plate body having bone plate threads and a bone plate head having one or more recesses, two or more layers in each of the one or more recesses, each of the two or more layers comprising a biocompatible material and a pharmaceutically effective amount of a pharmaceutical compound, at least one biocompatible barrier positioned between the at least two layers, and at least one biocompatible cap in each of the one or more recesses. Each of the one or more recesses on an implantable bone plate comprises an aperture, one or more lateral walls, and a base and wherein the two or more layers, the at least one biocompatible barrier, and the at least one biocompatible cap have the structure: biocompatible cap—(layer—biocompatible barrier)ₙ—layer—RECESS BASE, wherein n may be an integer from about 1 to about 100 and RECESS BASE is the base of the recess.

The present disclosure further relates to a spinal implant comprising a means for mechanically supporting, augmenting, or replacing one or more spinal structures; and a means for eluting a pharmaceutical agent to a subject's spine or a portion thereof.

### BRIEF DESCRIPTION OF THE DRAWINGS

Some embodiments of the disclosure may be understood by referring, in part, to the present disclosure and the accompanying drawings, wherein:

FIG. 1 illustrates a sectional side view of a spinal implant according to an example embodiment of the disclosure;

FIG. 2 illustrates a sectional side view of a spinal implant according to an example embodiment of the disclosure;

FIG. 3 illustrates a sectional side view of a spinal implant according to an example embodiment of the disclosure; and

FIG. 4 illustrates the concentration of a pharmaceutical compound as a function of time according to a specific example embodiment of the disclosure.

### DETAILED DESCRIPTION

The present disclosure relates, in some embodiments, to spinal implants (e.g., rigid spinal implants) configured and arranged to perform a mechanical function and a pharmacological function when placed in proximity with a spine. Mechanical functions may include, for example, connecting (e.g., rigidly and/or elastically) any spinal structure (e.g., bone, ligament, tendon, and/or cartilage) to any other structure (e.g., spinal, non-spinal, native, transplanted, and/or artificial), extending and/or limiting a spinal range of motion, and/or serving as a substitute (e.g. prosthetic) for a spinal structure (e.g., an artificial disc). Pharmacological functions may include, for example, any operation intended to contact a spine, spinal tissue, and/or spinal cell with a molecule intended to have a diagnostic and/or therapeutic effect. According to some embodiments, a spinal implant may comprise (a) a rigid spinal implant body having one or more recesses (e.g., crevices, fissures, gaps, holes, clefts, indentations, and/or the like); (b) one or more layers in each of the one or more recesses, each of the one or more layers comprising a biodegradable and/or biocompatible material and a pharmaceutically effective amount of a pharmaceutical compound; and (c) one or more biodegradable and/or biocompatible caps covering each of the one or more layers, wherein each biodegradable cap is configured and arranged to degrade at a slower or faster rate than the layer it covers, as desired and/or required by the application or situation. For example, if an implant develops a fibrous capsule on its surface, it may be desirably for one or more inner layers to degrade at a faster rate than the outer layers so as to release more drug at the site of therapy counteracting the increased diffusion barrier caused by the fibrous capsule.

A recess may have any regular or irregular geometric shape, according to some embodiments. For example, a recess may be cubical, hemispherical, conical, polyhedral, pyramidal, and/or cylindrical. A recess may include, in some embodiments, an aperture at the implant surface, one or more lateral walls, and a base. For example, a cylindrical base may have a circular aperture at one end, a single, continuous lateral wall (the body of the cylinder, and a flat, circular base or bottom. A recess aperture may be defined by the surrounding implant surface, may be circle-shaped, oval-shaped, square-shaped, rectangular, and/or triangular. The shape of a recess aperture may be the same as or different from the cross-section of a recess anywhere along its depth. A base of a recess may be round, beveled, or any other shape. Each recess may be from about one millimeter to about twenty millimeters in its longest transverse dimension (e.g., from about 0.5 millimeters to about 20 millimeters deep). The size and shape recesses in an implant may be the same, uniform (e.g., about the same), or different. A recess may be configured and arranged (e.g., positioned on an implant, sized, and/or shaped) to meter the release of a pharmaceutical compound. This may be either alone or in conjunction (e.g., cooperatively and/or synergistically) with the composition and/or structure of layers and/or barriers in the recess. In some embodiments, a recess may be a hole (e.g., having two or more apertures) and base (e.g., at the bottom of a U-shaped hole) or no base (e.g., a hole that spans the thickness of an implant). Each aperture of a hole may be capped (e.g., by a biodegradable cap).

In some embodiments, a spinal implant may be configured and arranged such that some or all of the recesses each comprise two or more layers. Some or all of the layers in these recesses may have at least one biodegradable barrier positioned between the at least two layers. A layer and/or a barrier may be configured and arranged (e.g., sized and formulated) to biodegrade (e.g., bioresorb, erode, decompose, and/or otherwise loose its integrity) at a desired rate. In some embodiments, a barrier may be configured and arranged to biodegrade at a rate that is faster than, the same as, or slower than another barrier, a cap, and/or a layer. Likewise, in some embodiments, a layer may be configured and arranged to biodegrade at a rate that is faster than, the same as, or slower than another layer, a cap, and/or a barrier. A cap similarly may be configured and arranged, according to some embodiments, to, biodegrade at a rate that is faster than, the same as, or slower than another cap, a layer, and/or a barrier. For example, some or all caps may be configured and arranged to biodegrade at the same rate as each other, but faster than some or all of the barriers.

In some embodiments, all recesses on an implant have the same or substantially the same composition. Some recesses may be configured and arranged to have a different composition than other recesses on the same implant. For example, it may be desirable to deliver one pharmaceutical to tissues on one side of a spinal plate and another pharmaceutical to the tissues on the other side of the plate. Recesses with the same composition may be located together in an area or areas of an implant or they may be interspersed with recesses of differing compositions. In some embodiments, it may be desirable to configure some of the recesses to release their contents quickly (e.g., within minutes or hours) and others to release their contents more slowly (e.g., hours, days, or weeks).

In some embodiments, adjacent barriers may be configured and arranged to “time” their degradation relative to each other. For example, if the biodegradable material comprises a copolymer of polylactide and glycolic acid, the erosion or degradation rate may be adjusted by varying the ratio of glycolic acid to lactic acid in the copolymer. In this example, the erosion rate may increase as the glycolid acid content increases. A first biodegradable barrier may have a composition ratio of 80:20, a second barrier may have a composition of 75:25, and a third barrier may have a composition of 70:30 of lactic acid to glycolic acid, representing barriers with increasing erosion rate and concomitant drug release rate.

In some embodiments, a barrier and/or cap may be pre-formed and/or cured in vivo and may be formulated accordingly. Examples of photo curable polymers include, without limitation, poly(anhydrides) comprising a mixed methacrylic anhydride of sebacic acid and 1,3 bis(p-carboxy phenoxy) propane.

Barriers and/or caps may comprise the same co-polymer composition, in some embodiments. It may be necessary and/or desirable, where barriers and/or caps have the same or similar compositions, in some embodiments, to activate biodegradation. Biodegradation may be activated, for example, by environment pH, by a drug, by contact with a body tissue, and/or by contact with a solvent. Biodegradation may be activated by one or more agents in an adjacent layer that move into the barrier and facilitate biodegradation, for example. Additionally, biodegradation may be activated by temperature, magnetic field, and/or radiation.

Each biodegradable layer independently may have the same or different composition. Each successive layer may be formulated to biodegrade at a rate that is faster than, the same as, or slower than an adjacent layer (e.g., an adjacent layer that is closer to the base of the recess). Each biodegradable barrier independently may have the same or different composition. Each successive barrier may be formulated to biodegrade at a rate that is faster than, the same as, or slower than an adjacent barrier (e.g., an adjacent barrier that is closer to the base of the recess). For example, each biodegradable layer may have the same composition and each biodegradable barrier may be biodegradable at a rate successively faster than the biodegradable barrier closer to the base of the recess. Up to all of the biodegradable barriers, for example, may be configured to biodegrade faster than their adjacent layers. In some embodiments, a barrier may be hydrophobic or hydrophilic, which may desirably speed or slow biodegradation.

According to some embodiments, biodegradable layers and barriers may be configured and arranged according to the following layer structure:

biodegradable cap-(layer-biodegradable barrier)ₙ-layer-RECESS,

wherein n may be an integer from about 1 to about 100 and RECESS is the base of the recess. For example, n may be from about 1 to about 5, from about 1 to about 10, from about 2 to about 10, from about 2 to about 20, from about 3 to about 10, from about 3 to about 30, from about 5 to about 25, and/or from about 2 to about 50. In some specific example embodiments, n may be 1, 2, 3, 4, 5, 6, 7, 8, 9, 10, 11, 12, 13, 14, 15, or more. According to some embodiments, a recess may be configured and arranged as a hole having biodegradable layers and barriers configured and arranged according to the following layer structure:

biodegradable cap-(layer-biodegradable barrier)ₙ-layer-biodegradable cap, wherein n may be an integer from about 1 to about 100 and/or as described above. In some embodiments, barriers and layers are uniformly interspersed. Barriers and layers, in some embodiments, may be non-uniformly dispersed. For example, two or more barriers may be adjacent without an intervening layer. Likewise, two or more layers may be adjacent to each other without an intervening barrier.

A spinal implant, according to some embodiments, may comprise any implant that supports, augments, and/or replaces one or more spinal structures. In some embodiments, a spinal implant may be situated in contact with and/or near a spinal structure. Non-limiting examples of spinal structures may include a vertebrae (e.g., vertebral end plate, vertebral foramen, vertebral body, cortical rim, cancellous, pedicle, spinous process, lamina, superior articular process, transverse process), an intervertebral disc, a sacrum, a coccyx, an annulus fibrosis, a nucleus pulposus, a spinal canal, a spinal cord, a ligamentum flavum, a posterior longitudinal ligament, a meninx (e.g., dura mater, arachnoid mater, pia mater), cerebrospinal fluid and/or any portion thereof. A spinal implant, according to some embodiments of the disclosure, may include all or a portion of a pedicle fixation system (e.g., a pedicle screw), a transforaminal lumbar interbody spacer, a thoracolumbar fixation system (e.g., a polyaxial screw), a thoracolumbar fixation system, (e.g., a pedicle screw), a posterior thoracolumbar fixation system, a transverse connector posterior spine implant (e.g., a pedicle screw), an anterior cervical plate system, a cervicothoracic fixation system (e.g., a polyaxial screw, an open hook, a rod, and/or a rod-to-rod connector), an occipital cervical fixation system, a cable fixation system (e.g., a cable and/or a cam), a bone plate system (e.g., a titanium bone plate and/or screw), an anterior lumbar system (e.g., a vertical endplate), a cage, an artificial disc, and/or an interbody spacer (e.g., a vertebral interbody spacer, a posterior lumbar interbody spacer and/or an expandable lumbar interbody spacer).

A cap, a barrier, and/or layer, in some embodiments, may comprise a biocompatible material, for example, a biodegradable material comprising a polymer. In some embodiments, a biocompatible material may not be biodegradable, yet it may release molecules contained within its matrix at a desired rate. A polymer may comprise, according to some embodiments, poly(MPC_{w}:LMAₓ:HPMA_{y}:TSMA_{z}), wherein MPC is 2 methacryoyloxyethylphosphorylcholine, LMA is lauryl methacrylate, HPMA is hydroxypropyl methacrylate, TSMA is trimethoxysilylpropyl methacrylate, and w, x, y, and z are independently integers from 0 to 60 (e.g., 23, 47, 25, and 5, respectively). A polymer may include, for example, one or more pendant phosphoryl groups (e.g., ammonium phosphate ester groups such as phosphoryl choline, glycerophosphoryl choline, analogues thereof, and/or combinations thereof). A polymer may comprise a compound selected from the group consisting of a phosphorylcholine linked macromolecule, an oligoethylenimine, a polyethylenimine, and/or combinations thereof. In some embodiments, a polymer may comprise a material selected from a polycaprolactone, a poly-D,L-lactic acid, a poly-L-lactic acid, a poly(lactide-co-glycolide), a poly(hydroxybutyrate), a poly(hydroxybutyrate-co-valerate), a polydioxanone, a polyorthoester, a polyanhydride, a poly(glycolic acid), a poly(glycolic acid-co-trimethylene carbonate), a poly(methyl methacrylate) (PMMA), a polyphosphoester, a polyphosphoester urethane, a poly(amino acid) (e.g., polypeptide and/or protein), cyanoacrylates, a poly(trimethylene carbonate), a poly(iminocarbonate), a polyalkylene oxalate, a polyphosphazene, a polyiminocarbonate, an aliphatic polycarbonate, a fibrin, a fibrinogen, a cellulose, a starch, a collagen, a Parylene®, a Parylast®, and combinations thereof. A polymer, in some embodiments, may comprise a material selected from a polyurethane. Examples of a polyurethane may include, for example, a polycarbonate urethane, a polyethylene, a polyethylene terapthalate, an ethylene vinyl acetate, an ethylene vinyl alcohol, a silicone (e.g., a polysiloxane and/or a substituted polysiloxane), a polyethylene oxide, a polybutylene terepthalate-co-PEG, a PCL-co-PEG, a PLA-co-PEG, a polyacrylate, a polyvinyl pyrrolidone, a polyacrylamide, and combinations thereof. Non-limiting examples of other suitable polymers include thermoplastic elastomers in general, polyolefin elastomers, EPDM rubbers and polyamide elastomers, and biostable plastic material such as acrylic polymers, and its derivatives, nylon, polyesters and epoxies.

A pharmaceutical compound, according to some embodiments, may comprise a compound selected from the group consisting of an analgesic, an antimicrobial agent, an anti-inflammatory agent, a nucleic acid, a fibrosis-inducing agent (e.g., an adhesive, an arterial vessel wall irritant, a bone morphogenic protein, an extracellular matrix component, an inflammatory cytokine, a polymer, and combinations thereof), and combinations thereof. A fibrosis-inducing agent may be selected from the group consisting of crosslinked poly(ethylene glycol)-methylated collagen, a cyanoacrylate, a crystalline silicate, copper, ethanol, metallic beryllium, an oxide of metallic beryllium, neomycin, quartz dust, silica, silk, talc, talcum powder, wool, bleomycin, bone morphogenic protein-2, bone morphogenic protein-3, bone morphogenic protein-4, bone morphogenic protein-5, bone morphogenic protein-6, bone morphogenic protein-7, connective tissue growth factor, collagen, fibrin, fibrinogen, fibronectin, basic fibroblast growth factor, granulocyte-macrophage colony stimulating factor, growth hormones, insulin growth factor-1, interleukin-1, interleukin-6, interleukin-8, nerve growth factor, platelet-derived growth factor, transforming growth factor-beta, tumor necrosis factor alpha, vascular endothelial growth factor, leptin, chitosan, N-carboxybutylchitosan, a poly(alkylcyanoacrylate), poly(ethylene-co-vinylacetate), poly(ethylene terephthalate), a polylysine, polytetrafluoroethylene, a polyurethane, an RGD protein, vinyl chloride, and combinations thereof. A pharmaceutical compound may be present, according to some embodiments, in liquid, viscous, gel, and/or solid form (e.g. solid particles). For example, a pharmaceutical compound may be integrated into a layer as a solid or powder. In some embodiments, a pharmaceutical compound may be loaded in a nanostructure (e.g., a nanotube) prior to integration into a layer. A nanostructure may be biodegradable or non-biodegradable.

In some embodiments, the present disclosure relates to methods for delivering a molecule intended to have a diagnostic and/or therapeutic effect (e.g., a pharmaceutical compound) to a spine (e.g., in a subject). For example, a method of delivering a molecule intended to have a diagnostic and/or therapeutic effect may comprise implanting in the subject's spine a spinal implant. The present disclosure also relates, in some embodiments, to methods of manufacturing a spinal implant. For example, a method of manufacturing a spinal implant may include (a) forming a rigid spinal implant body having at least one recess, (b) depositing in each recess one or more layers comprising a biodegradable material and a pharmaceutical compound, wherein the layer is configured and arranged to deliver a pharmaceutically effective amount of the pharmaceutical compound to the subject's spine; and (c) optionally depositing in each recess one or more barriers comprising a biodegradable material; and/or (d) depositing in each recess at least one biodegradable cap over the one or more layers obturating the recess, wherein each biodegradable cap is configured and arranged to biodegrade at a rate that is faster than, about the same as, of slower rate than the layer it covers.

Depositing a cap, barrier, and/or layer in a recess may comprise applying, loading, coating, covering, injecting, printing, depositing, and/or otherwise placing a material on or in an implant (e.g., in a well). For example, an implant may be dip-coated with a material and excess material optionally may be removed (e.g., wiped, washed) from the surface. A material may be deposited in a spinal implant well by printing (e.g., using an ink-jet printer) according to some embodiments. For example, a method of manufacturing a spinal implant may include (a) providing a fluid-dispenser (e.g., a fluid-jetting device) having a dispensing element operable to dispense a material (e.g., a cap, a barrier, a layer) in discrete droplets, wherein each droplet has a controlled trajectory, (b) creating relative movement between the dispensing element and the prosthesis to define a dispensing path, and (c) selectively dispensing the material from the fluid-dispenser in a raster format to a predetermined portion of the spinal implant along the dispensing path. In some embodiments, a material may be selectively dispensed from a dispensing element to a predetermined portion of a spinal implant in a raster format along a dispensing path. In some embodiments, a cap, a barrier, and/or a layer, may be deposited in a recess in one or more passes by a fluid dispenser (e.g., ink-jet printer head). The thickness and/or amount of material in a cap, barrier, and/or layer may be controlled, in some embodiments, by the number of passes and/or the amount of material dispensed (e.g., drop size).

A raster format may be a continuous or non-continuous dispensing pattern of droplets of material dispensed at specific intervals. The relative motion of the dispensing element and the spinal implant to be loaded with beneficial agent creates a dispensing path which includes a sequential series of linear parallel passes that traverse back and forth along one axis of the spinal implant. The relative motion may be continued in a linear manner between forward and backward or right to left and left to right or upward and downward, depending on the frame of reference. A traversal or a pass is completed when the relative motion reverses direction. That is, relative motion continues past the spinal implant, and then decelerates, stops, reverses direction and accelerates to a constant velocity. After each pass, the position of the dispensing element or spinal implant relative to the dispensing element may be changed or incremented such that additional droplets do not impact in the same location during the subsequent pass. Some overlap may be permitted in some embodiments. Areal density of droplets may be varied in a systematic way according to some embodiments. A fluid dispenser may be used in combination with a detector operable to detect the location of a recess and a controller that is operable to receive input from the detector and direct the position and/or material output of the fluid dispenser.

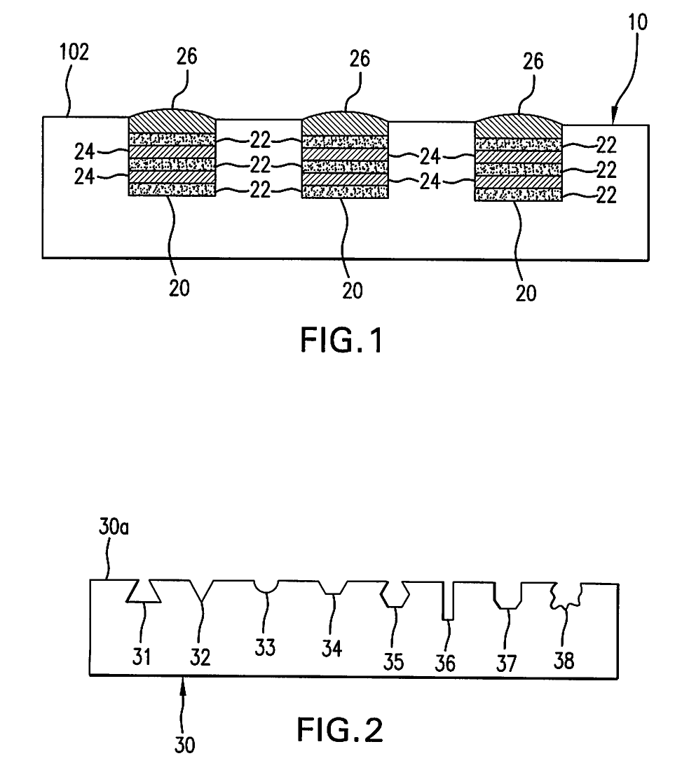

FIG. 1 shows a sectional side view of a spinal implant according to a specific example embodiment of the disclosure. Plate 10 comprises 3 recesses 20, which are filled with biodegradable materials, specifically, layers 22, barriers, 24, and caps 26. The surface of caps 26 may be flat (not shown) or slightly domed relative to implant surface 10a.

FIG. 2 shows a sectional side view of a spinal implant according to a specific example embodiment of the disclosure. Rod 30 comprises recesses 31, 32, 33, 34, 35, 36, 37, and 38 having a variety of shapes. Apertures for each of the recesses are defined by implant surface 30a.

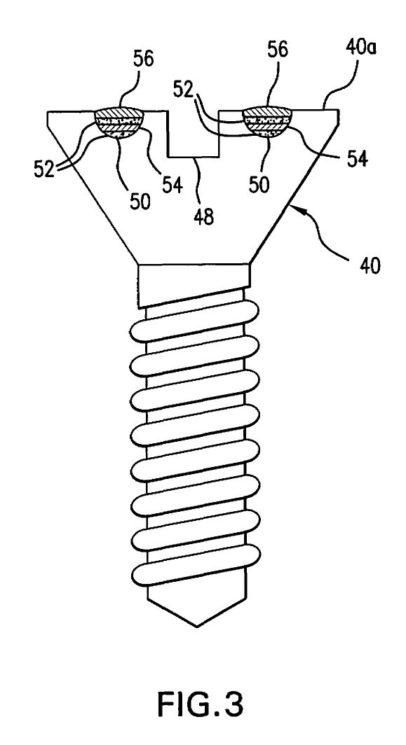

FIG. 3 shows a sectional side view of a spinal implant according to a specific example embodiment of the disclosure. Screw 40 comprises 2 recesses 50, which are filled with biodegradable materials, specifically, layers 52, barriers, 54, and caps 56. Screw slot 48 for securing the screw in bone using a tool (e.g., a screwdriver) may not contain biodegradable materials. The surface of caps 56 may be flat (not shown) or slightly domed relative to implant surface 40a.

As will be understood by those skilled in the art who have the benefit of the instant disclosure, other equivalent or alternative devices, systems, and methods for delivering a pharmaceutical compound to a spine can be envisioned without departing from the essential characteristics thereof. Accordingly, the manner of carrying out the disclosure as shown and described are to be construed as illustrative only.

Persons skilled in the art may make various changes in the shape, size, number, and/or arrangement of parts without departing from the scope of the instant disclosure. For example, a recess may be proportioned such that it does not compromise the structural integrity and/or structural function of an implant. Also, where ranges have been provided, the disclosed endpoints may be treated as exact and/or estimates as desired or demanded by the particular embodiment In addition, it may be desirable in some embodiments to mix and match range endpoints. A pharmaceutical compound may be deposited on an implant and/or layer and/or mixed into a layer material by any available method. In addition, a biodegradable material may be deposited on an implant and/or mixed into a cap, layer, and/or barrier by any available method. For example, a biodegradable material may be applied, printed, and/or coated (e.g., sprayed or spray-dried) onto an implant. These equivalents and alternatives along with obvious changes and modifications are intended to be included within the scope of the present disclosure. Accordingly, the foregoing disclosure is intended to be illustrative, but not limiting, of the scope of the disclosure as illustrated by the following claims.

### EXAMPLES

Some specific example embodiments of the disclosure may be illustrated by one or more of the examples provided herein.

### Example 1

### Kinetics of Controlled Drug Release

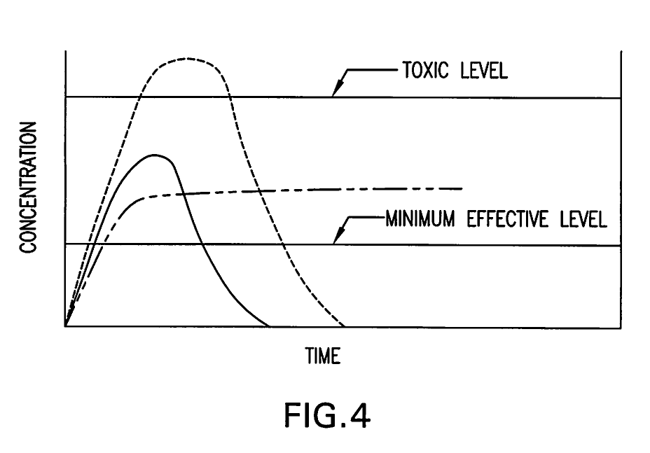

FIG. 4 illustrates a plot of drug concentration as a function of time. Typical results obtained using existing methods of drug therapy are represented as the dashed and solid traces. As shown, these methods may result in a spike of drug release that exceeds toxic levels. Alternatively, these methods may only achieve therapeutic levels for a limited time. By contrast, some embodiments of the present disclosure may permit controlled release of a pharmaceutical compound may achieve a sustained therapeutic level as represented by the dashed-dotted trace.

### Example 2

### Ink-Jet Coating

A cap, a layer, and/or a barrier, may be applied to a stent (e.g., deposited in a recess) by ink-jet printing. Stainless steel stents made from 316 stainless steel and stent coating polymer were obtained from Biocompatibles Ltd. Farnham, U.K. The coating polymer was a phosphorylcholine-linked methacrylate tetracopolymer made from four monomers represented as follows, poly(MPC_{w}:LMAₓ:HPMA_{y}:TSMA_{z}), wherein MPC is 2 methacryoyloxyethylphosphorylcholine, LMA is lauryl methacrylate, HPMA is hydroxypropyl methacrylate, TSMA is trimethoxysilylpropyl methacrylate. It is referred subsequently as “PC polymer.” The stent surfaces had a coating of PC polymer which had been deposited on the stent by dip-coating and cured, prior to the application of drug by reagent printing. The polymer precoat was employed to increase adhesion between the drug and the stent and thus eliminate potential release bursts. Dip-coating ensured a continuous polymer film around the struts.

Additional PC polymer was sometimes dissolved in the drug solutions for jetting up to the level of 10% by weight with respect to drug, to serve as binder. The jetting solvent for both of the drugs (fenofibrate and ABT-578, a rapamycin derivative) and coating polymer was reagent grade isobutanol.

### Example 3

### Loading the Drug Delivery Recess on a Spinal Implant to Stimulate Bone Formation

Implants similar to that illustrated in FIG. 1 may be placed on a conveyor belt in defined locations. The conveyor belt may travel through a drying tunnel used to facilitate the loading process. The location of the implant and the location and dimensions of the recess, often referred to as the drug or delivery system addresses may be entered into a program that controls the dispensing of drugs and drug delivery systems along the conveyor belt. The dispensing of drug and/or drug+delivery system can be done with a reagent-jetter which uses the principles of an ink-jet printer to produce droplets of known size and number. For this example, three printer heads coupled independently to three liquid reagent vials may be used. Vial 1 may contain a therapeutic agent, for example, an aqueous solution of plasmid DNA coding for the production of a bone morphogenetic protein (BMP), such as BMP-2. Vial 2 may contain polylactice/glycolide copolymer dissolved in methylene chloride or alternatively chloroform. Vial 3 may contain a transfection agent, for example, polyethyleneimine 25,000 molecular weight dissolved in water. To start the process, the empty device may pass over the printer head coupled to Vial 1, containing the DNA solution and known amount is jetted into the recess as they pass by into the drying tunnel. After exiting the drying tunnel, the second pass employs the second print head, which is coupled to Vial 2, that dispenses a layer of polylactide/glycolide, followed by transit of the device through the drying tunnel a second time. Upon exit from the drying tunnel, the device may interact with a third print head coupled to vial 3, which dispenses the transfection agent. After passing through the drying tunnel for the third time, the sequence of dispenses is repeated for a sufficient number of times to give the desired drug dosage and duration of drug release. Finally a capping layer of polylactide/glycolide may be applied to each recess.

### Example 4

### Loading the Drug Delivery Recess on a Spinal Implant to Reduce Inflammation

According to this example, loading may occur as described in Example 3, except that vial 3 and print head 3 are not present. In addition, Vial 1 contains dexamethazone dissolved in methylene chloride or alternatively chloroform. Vial 2 contains polylactice/glycolide copolymer dissolved in methylene chloride or alternatively chloroform. The dispense and drying process may be repeated for a sufficient number of times. Finally, a capping layer of polylactide/glycolide may be applied to each recess.

If a burst of drug is desired at the time of implantation, the capping layer may be omitted. Alternatively, a capping layer may be made by jetting both drug and polymer solution into the recess prior to entering the drying tunnel.

### Example 5

### Loading the Drug Delivery Recess on a Spinal Implant to Reduce Inflammation and Stimulate Bone Growth

In this example, elements of Example 3 and Example 4 are used. For a selected number of recess, dexamethazone plus drug release layers are dispensed. For the remainder of the recesses, DNA followed by drug release layer followed by tranfection agent may be dispensed. A final capping layer can be deposited on all of the recess or omitted if a burst effect is desired.

What is claimed is:

1. A spinal implant, said implant comprising:
a rigid spinal implant body having one or more recesses;
one or more layers in each of the one or more recesses, each of the one or more layers comprising a biocompatible material and a pharmaceutically effective amount of a pharmaceutical compound; and
at least one biocompatible cap in each of the one or more recesses.

2. A spinal implant according to claim 1, wherein each of the one or more recesses independently has a shape selected from the group consisting of cube, hemisphere, cone, polyhedron, pyramid, and cylinder.

3. A spinal implant according to claim 1, wherein each of the one or more recesses comprises an aperture, one or more lateral walls, and a base.

4. A spinal implant according to claim 1 further comprising two or more layers in each of the one or more recesses.

5. A spinal implant according to claim 4, wherein the spinal implant further comprises at least one biocompatible barrier positioned between the at least two layers.

6. A spinal implant according to claim 5, wherein each of the one or more recesses comprises an aperture, one or more lateral walls, and a base and wherein the two or more layers, the at least one biocompatible barrier, and the at least one biocompatible cap have the structure:
biocompatible cap—(layer—biocompatible barrier)ₙ—layer—RECESS BASE, wherein n may be an integer from about 1 to about 50 and RECESS BASE is the base of the recess.

7. A spinal implant according to claim 6, wherein each of the one or more layers has the same composition.

8. A spinal implant according to claim 6, wherein n is from 2 to about 50.

9. A spinal implant according to claim 8, wherein each biocompatible barrier is biodegradable at a rate successively faster than the biocompatible barrier closer to the base of the recess.

10. A spinal implant according to claim 8, wherein each biocompatible barrier is biodegradable at a faster rate than each of the one or more layers.

11. A spinal implant according to claim 1, wherein the rigid spinal implant body comprises a pedicle screw, a polyaxial screw, an interbody spacer, an anterior cervical plate, an open hook, a rod, a rod-to-rod connector, a cable, a cam, a bone plate, a bone screw, a vertical endplate, a cage, an artificial disc, and combinations thereof.

12. A spinal implant according to claim 1, wherein each recess is from about one millimeter to about twenty millimeters in its longest transverse dimension.

13. A spinal implant according to claim 1, wherein each recess is from about 0.5 millimeters to about 20 millimeters deep.

14. A spinal implant according to claim 1, wherein the biocompatible material comprises a polymer.

15. A spinal implant according to claim 14, wherein the polymer comprises a macromolecule comprising a pendant phosphorylcholine group.

16. A spinal implant according to claim 14, wherein the polymer comprises poly(MPC_{w}:LMAₓ:HPMA_{y}:TSMA_{z}), wherein MPC is 2 methacryoyloxyethylphosphorylcholine, LMA is lauryl methacrylate, HPMA is hydroxypropyl methacrylate, TSMA is trimethoxysilylpropyl methacrylate, and w, x, y, and z are independently integers from 0 to 60.

17. A spinal implant according to claim 14, wherein the polymer comprises a compound selected from the group consisting of a phosphorylcholine linked macromolecule, an oligoethylenimine, a polyethylenimine, and combinations thereof.

18. A spinal implant according to claim 1, wherein the pharmaceutical compound comprises a compound selected from the group consisting of an adhesive, an arterial vessel wall irritant, a bone morphogenic protein, an extracellular matrix component, an inflammatory cytokine, a polymer, and combinations thereof.

19. A spinal implant according to claim 1, wherein the pharmaceutical compound comprises a compound selected from the group consisting of an analgesic, an antimicrobial agent, an anti-inflammatory agent, a fibrosis-inducing agent, and combinations thereof.

20. A method of delivering a pharmaceutically effective amount of a pharmaceutical compound to a subject's spine, said method comprising:
implanting in the subject's spine a spinal implant comprising:
a rigid spinal implant body having one or more recesses;
one or more layers in each of the one or more recesses, each of the one or more layers comprising a biocompatible material and a pharmaceutical compound, wherein the layer is configured and arranged to deliver a pharmaceutically effective amount of the pharmaceutical compound to the subject's spine; and
at least one biocompatible cap in each of the one or more recesses.

21. A method of delivering a pharmaceutically effective amount of a pharmaceutical compound to a subject's spine according to claim 20, wherein the spinal implant further comprises two or more layers in each of the one or more recesses.

22. A method of delivering a pharmaceutically effective amount of a pharmaceutical compound to a subject's spine according to claim 21, wherein the spinal implant further comprises at least one biocompatible barrier positioned between the at least two layers.

23. A method of delivering a pharmaceutically effective amount of a pharmaceutical compound to a subject's spine according to claim 22, wherein each of the one or more recesses comprises an aperture, one or more lateral walls, and a base and wherein the two or more layers, the at least one biocompatible barrier, and the at least one biocompatible cap biocompatible have the structure:
biocompatible cap—(layer—biocompatible barrier)ₙ—layer—RECESS BASE, wherein n may be an integer from about 1 to about 100 and RECESS BASE is the base of the recess.

24. A method of delivering a pharmaceutically effective amount of a pharmaceutical compound to a subject's spine according to claim 23, wherein each biocompatible layer has the same composition.

25. A method of delivering a pharmaceutically effective amount of a pharmaceutical compound to a subject's spine according to claim 24, wherein n is from 2 to about 50.

26. A method of delivering a pharmaceutically effective amount of a pharmaceutical compound to a subject's spine according to claim 25, wherein each biocompatible barrier is biodegradable at a rate successively faster than the biocompatible barrier closer to the base of the recess.

27. A method of delivering a pharmaceutically effective amount of a pharmaceutical compound to a subject's spine according to claim 20, wherein the rigid spinal implant body comprises a pedicle screw, a polyaxial screw, an interbody spacer, an anterior cervical plate, an open hook, a rod, a rod-to-rod connector, a cable, a cam, a bone plate, a bone screw, a vertical endplate, a cage, an artificial disc, and combinations thereof.

28. A method of delivering a pharmaceutically effective amount of a pharmaceutical compound to a subject's spine according to claim 20, wherein each recess is from about one millimeter to about twenty millimeters in its longest transverse dimension.

29. A method of delivering a pharmaceutically effective amount of a pharmaceutical compound to a subject's spine according to claim 20, wherein each recess is from about 0.5 millimeters to about 20 millimeters deep.

30. A method of delivering a pharmaceutically effective amount of a pharmaceutical compound to a subject's spine according to claim 20, wherein the biocompatible material comprises a polymer.

31. A method of delivering a pharmaceutically effective amount of a pharmaceutical compound to a subject's spine according to claim 30, wherein the polymer comprises a macromolecule comprising a pendant phosphorylcholine group.

32. A method of delivering a pharmaceutically effective amount of a pharmaceutical compound to a subject's spine according to claim 30, wherein the polymer comprises poly(MPC_{w}:LMAₓ:HPMA_{y}:TSMA_{z}) wherein MPC is 2 methacryoyloxyethylphosphorylcholine, LMA is lauryl methacrylate, HPMA is hydroxypropyl methacrylate, TSMA is trimethoxysilylpropyl methacrylate, and w, x, y, and z are independently integers from 0 to 60.

33. A method of delivering a pharmaceutically effective amount of a pharmaceutical compound to a subject's spine according to claim 30, wherein the polymer comprises a compound selected from the group consisting of a phosphorylcholine linked macromolecule, an oligoethylenimine, a polyethylenimine, and combinations thereof.

34. A method of delivering a pharmaceutically effective amount of a pharmaceutical compound to a subject's spine according to claim 20, wherein the pharmaceutical compound comprises a compound selected from the group consisting of an adhesive, an arterial vessel wall irritant, a bone morphogenic protein, an extracellular matrix component, an inflammatory cytokine, a polymer, and combinations thereof.

35. A method of delivering a pharmaceutically effective amount of a pharmaceutical compound to a subject's spine according to claim 20, wherein the pharmaceutical compound comprises a compound selected from the group consisting of an analgesic, an antimicrobial agent, an anti-inflammatory agent, a fibrosis-inducing agent, and combinations thereof.

36. A method of manufacturing a spinal implant, said method comprising:
providing a rigid spinal implant body having one or more recesses;
depositing in each of the one or more recesses at least one layer comprising a biocompatible material and a pharmaceutical compound, wherein the layer is configured and arranged to deliver a pharmaceutically effective amount of the pharmaceutical compound to the subject's spine; and
depositing in each of the one or more recesses at least one biocompatible cap over the one or more layers obturating the recess, wherein each biocompatible cap is configured and arranged to biodegrade at a slower rate than the layer it covers.

37. A method of manufacturing a spinal implant according to claim 36, wherein the depositing in each of the one or more recesses the one or more layers comprising a biocompatible material and a pharmaceutical compound further comprises printing the one or more layers.

38. A method of manufacturing a spinal implant according to claim 36 further comprising two or more layers in each of the one or more recesses.

39. A method of manufacturing a spinal implant according to claim 38, wherein the spinal implant further comprises at least one biocompatible barrier positioned between the two or more layers.

40. A method of manufacturing a spinal implant according to claim 39, wherein each of the one or more recesses comprises an aperture, one or more lateral walls, and a base and wherein the two or more layers, the at least one biocompatible barrier, and the at least one biocompatible cap have the structure:
biocompatible cap—(layer—biocompatible barrier)ₙ—layer—RECESS BASE, wherein n may be an integer from about 1 to about 50 and RECESS BASE is the base of the recess.

41. A method of manufacturing a spinal implant according to claim 40, wherein each of the one or more layers has the same composition.

42. A method of manufacturing a spinal implant according to claim 40, wherein n is from 2 to about 50.

43. A method of manufacturing a spinal implant according to claim 40, wherein each biocompatible barrier is biodegradable at a rate successively faster than the biocompatible barrier closer to the base of the recess.

44. A method of manufacturing a spinal implant according to claim 40, wherein each biocompatible barrier is biodegradable at a faster rate than each of the one or more layers.

45. A method of manufacturing a spinal implant according to claim 36, wherein the rigid spinal implant body comprises a pedicle screw, a polyaxial screw, an interbody spacer, an anterior cervical plate, an open hook, a rod, a rod-to-rod connector, a cable, a cam, a bone plate, a bone screw, a vertical endplate, a cage, an artificial disc, and combinations thereof.

46. A method of manufacturing a spinal implant according to claim 36, wherein each of the one or more recesses is from about one millimeter to about twenty millimeters in its longest transverse dimension.

47. A method of manufacturing a spinal implant according to claim 36, wherein each of the one or more recesses is from about 0.5 millimeters to about 20 millimeters deep.

48. A method of manufacturing a spinal implant according to claim 36, wherein the biocompatible material comprises a polymer.

49. A method of manufacturing a spinal implant according to claim 48, wherein the polymer comprises a macromolecule comprising a pendant phosphoryicholine group.

50. A method of manufacturing a spinal implant according to claim 48, wherein the polymer comprises poly(MPC_{w}:LMAₓ:HPMA_{y}:TSMA_{z}), wherein MPC is 2 methacryoyloxyethylphosphorylcholine, LMA is lauryl methacrylate, HPMA is hydroxypropyl methacrylate, TSMA is trimethoxysilylpropyl methacrylate, and w, x, y, and z are independently integers from 0.5 to 60.

51. A method of manufacturing a spinal implant according to claim 48, wherein the polymer comprises a compound selected from the group consisting of a phosphorylcholine linked macromolecule, an oligoethylenimine, a polyethylenimine, and combinations thereof.

52. A method of manufacturing a spinal implant according to claim 36, wherein the pharmaceutical compound comprises a compound selected from the group consisting of an adhesive, an arterial vessel wall irritant, a bone morphogenic protein, an extracellular matrix component, an inflammatory cytokine, a polymer, and combinations thereof.

53. A method of manufacturing a spinal implant according to claim 36, wherein the pharmaceutical compound comprises a compound selected from the group consisting of an analgesic, an antimicrobial agent, an anti-inflammatory agent, a fibrosis-inducing agent, and combinations thereof.

54. An implantable bone screw comprising:
a rigid implantable bone screw body having bone screw threads and a bone screw head having one or more recesses;
two or more layers in each of the one or more recesses, each of the two or more layers comprising a biocompatible material and a pharmaceutically effective amount of a pharmaceutical compound;
at least one biocompatible barrier positioned between the at least two layers; and
at least one biocompatible cap in each of the one or more recesses.

55. An implantable bone screw according to claim 54, wherein the bone screw is configured and arranged as a pedicle screw or a polyaxial screw.

56. An implantable bone screw according to claim 54, wherein each of the one or more recesses comprises an aperture, one or more lateral walls, and a base and wherein the two or more layers, the at least one biocompatible barrier, and the at least one biocompatible cap have the structure:
biocompatible cap—(layer—biocompatible barrier)ₙ—layer—RECESS BASE, wherein n may be an integer from about 1 to about 100 and RECESS BASE is the base of the recess.

57. An implantable bone plate comprising:
a rigid implantable bone plate body having one or more recesses;
two or more layers in each of the one or more recesses, each of the two or more layers comprising a biocompatible material and a pharmaceutically effective amount of a pharmaceutical compound;
at least one biocompatible barrier positioned between the at least two layers; and
at least one biocompatible cap in each of the one or more recesses.

58. An implantable bone plate according to claim 57, wherein the bone plate is configured and arranged as a cervical plate.

59. An implantable bone plate according to claim 57, wherein each of the one or more recesses comprises an aperture, one or more lateral walls, and a base and wherein the two or more layers, the at least one biocompatible barrier, and the at least one biocompatible cap have the structure:
biocompatible cap—(layer—biocompatible barrier)ₙ—layer—RECESS BASE, wherein n may be an integer from about 1 to about 100 and RECESS BASE is the base of the recess.

60. A spinal implant comprising:
a means for mechanically supporting, augmenting, or replacing one or more spinal structures; and
a means for eluting a pharmaceutical agent to a subject's spine or a portion thereof.
